(19) United States
(12) Patent Application Publication
(10) Pub. No.: US 2025/0353771 A1
(43) Pub. Date: Nov. 20, 2025

(54) LOW-EMISSION GENERATION OF RENEWABLE BIOHYDROGEN AND BIOMETHANE FROM ORGANIC WASTE

(71) Applicant: ITility, L.L.C., Chantilly, VA (US)

(72) Inventors: Peter SLOAD, Washington, VA (US); Erik OVERBY, Purcellville, VA (US); Benjamin FRANK, Germantown, MD (US); Kevin LAUER, Front Royal, VA (US); Michael SIBILO, Chantilly, VA (US)

(73) Assignee: ITility, L.L.C., Chantilly, VA (US)

(21) Appl. No.: 19/289,556

(22) Filed: Aug. 4, 2025

Related U.S. Application Data
(63) Continuation-in-part of application No. 17/863,868, filed on Jul. 13, 2022, now Pat. No. 12,404,199.
(60) Provisional application No. 63/678,662, filed on Aug. 2, 2024.

(51) Int. Cl. C02F 11/04; C02F 11/18; C12M 1/00; C12M 1/107; C12M 3/00; C12P 5/02

(52) U.S. Cl. CPC C02F 11/04; C02F 11/18; C12M 21/04; C12M 23/44; C12M 23/58; C12M 31/00; C12P 5/023; C02F 2201/007; C02F 2303/26

(57) ABSTRACT

There is disclosed a system, method and apparatus for generating renewable energy from common waste streams in a low-carbon manner. This system is modular and applicable to operations of a range of sizes. The system comprises a waste homogenization system; a feedstock preparation component involving dilution, nutrient adjustment, and mixing; a pretreatment tank; a hydrolysis tank; and an optional photosynthetic bioreactor. Through use of the system, organic waste is converted into biohydrogen (H2) and/or biomethane (CH4). The choice between producing each gas individually or in combination is controlled via selective treatment of the incoming waste.

### RELATED APPLICATION

This application claims the benefit of U.S. Provisional Application Ser. No. 63/678,662, filed Aug. 2, 2024, entitled “Low-Emission Generation Of Renewable Biohydrogen And Biomethane From Organic Waste,” and is a continuation-in-part of U.S. application Ser. No. 17/863,868, filed Jul. 13, 2022, entitled “Modular Anaerobic Digestion Point-Of-Waste Renewable Energy Apparatus And Method,” which applications are incorporated herein by reference in their entirety.

### FIELD OF THE INVENTION

The present invention relates to the low-emission generation of renewable biohydrogen and biomethane from organic waste. More particularly, the present invention is an innovative system designed for the efficient conversion of organic waste into renewable energy, specifically targeting the production of biohydrogen and biomethane through anaerobic fermentation processes. This system is uniquely tailored for waste collection from organizations such as restaurants, farms, and municipalities which have the goal of fostering sustainable waste management practices. The invention's utility and advantages include the combined generation of biohydrogen and biomethane from organic waste while also benefitting from the novel integration of a photosynthetic bioreactor which contributes a significant reduction in carbon emissions via carbon sequestration. The modularity and scalability of the system allows it to benefit a range of users and process a variety of organic waste streams, thus increasing the availability of a carbon sequestration technology to a multitude of differently structured organizations. Through use of the system, organic waste is microbially converted into biohydrogen, a carbon-free fuel source which only generates water when burnt. Organic waste is processed, converted into a feedstock slurry, treated, and digested in a single system. The system can operate to support the production of either biohydrogen or biomethane, or alternatively can be affixed to an anaerobic digester (“AD”) to allow both gases to be produced in concert. Selection for biohydrogen is made by treating the slurry with an acidic or thermal treatment to eliminate methanogens from the feedstock. If biomethane is desired, this treatment is unnecessary. Each gas is optionally processed in a generator or combined heat and power (“CHP”) engine to generate heat, electricity, or both. Emissions from the system are further reduced via the affixation of a photosynthetic bioreactor which enables carbon capture. After digestion, the slurry is dried into nutrient-rich digestate which is usable as a fertilizer, an especially valuable product for owners who utilize crops such as restaurants, cafeterias, farms, and groceries. Through its innovative design and functional capabilities, this invention represents a significant advancement in the field of waste-to-energy technologies and offers a practical solution for reducing carbon footprints and promoting sources of renewable energy.

### BACKGROUND OF THE INVENTION

The invention presents several improvements compared to existing technologies in the domain of waste-to-energy conversion systems. The system's modular design allows for the integration of both an AD and a photosynthetic bioreactor, facilitating the production of a low-carbon energy product. This modularity and flexibility enables customization based on specific waste processing and energy generation needs between different projects. The system is designed for simplicity, making it accessible to individuals without specialized technical knowledge, thereby broadening its applicability across various scales of operation. The system is especially well-suited for businesses of any size aiming to process their waste in a manner that is both environmentally friendly and effective at lowering carbon emissions. The system's components are defined at a much higher level of detail than the prior art making the construction of the system simpler, easier and at a lower cost than prior art biohydrogen systems. This allows a much wider range of users to convert their waste into low-carbon renewable energy via biohydrogen production. In addition, the nutrient-rich algae and digestate byproducts equip users with a number of downstream options for promoting crop and animal health or product sales at their associated operations. This encompasses a means of organic waste disposal that circumvents the emissions associated with landfilling and other traditional methods while also directly benefiting the user's organization.

Accordingly, the disclosed invention provides improvements over the prior art systems, apparatus and methods and the shortcomings thereof.

### SUMMARY OF THE INVENTION

The invention is directed to an advanced system capable of generating renewable energy from common waste streams in a low-carbon manner. This system is applicable to operations of a range of sizes with the goal of enabling its adoption across a wide market. Included are a waste homogenization system, a feedstock preparation component involving dilution, nutrient adjustment, and mixing, a pretreatment tank, a hydrolysis tank (H-tank), and an optional photosynthetic bioreactor. Ancillary system components include an optional anaerobic digester component, gas treatment lines, a dewatering component, and an optional CHP generator as well as the plumbing and piping required to move liquid and/or gas between each component. Through use of the system, organic waste is converted into biohydrogen (H2), a carbon-free fuel source, and/or biomethane (CH4). The choice between producing each gas individually or in combination is controlled via selective treatment of the incoming waste. If biohydrogen is desired, the waste is subjected to an acid or heat treatment, while this is not required if biomethane alone is the goal. Equipping the system with an AD allows both biohydrogen and biomethane to be produced. Regardless, either gaseous product can be processed in a generator or CHP engine to generate heat, electricity, or both. Carbon emissions produced by microbes or from energy generation in the form of CO2 are reduced by running exhaust gas through an algae bed where they are captured as additional biomass.

System operation begins upon receipt of organic waste which is soon thereafter converted into a feedstock slurry suitable for microbial degradation via a combination of particle size reduction, dilution, nutrient adjustment, and mixing. The feedstock slurry is then pumped into the pretreatment tank where it is exposed to acidic or thermal conditions which deactivate methanogenic microbes in the waste. If biomethane alone is desired, this step is skipped. The slurry is then transferred into the H-tank where it is held at a pH of under 6 and mesophilic temperatures over the span of 3-7 days to generate biohydrogen. If biomethane alone is desired, this step is skipped. After biohydrogen is produced, the slurry can then be either transferred into an adjoining anaerobic digester or passed to a dewatering system to generate a useful nutrient-rich digestate byproduct. If passed to an AD, biomethane is generated in addition to the previous biohydrogen product produced in the hydrolysis tank. If biomethane alone is desired, the slurry is pumped directly into the AD from the start of the process without pretreatment and bypassing the H-tank. Alternatively, the H-tank can have its conditions set to support methane production in place of an AD. Both tanks can be either operated in a batch mode where discrete loads of feedstock are fully digested before fully emptying and refilling the tank, or in continuous mode where aliquots of slurry are replaced daily to keep the ratio of fresh and digesting feedstock in balance.

Once either gaseous product is produced, it passes through a gas treatment line where it is conditioned for use as a source of renewable energy. Gaseous components including any or all of hydrogen sulfide (H2S), water (H2O), carbon dioxide (CO2), siloxanes, or other species are removed by a combination of coolers, filters, etc. to yield a pure product. The final gas is either biohydrogen or biomethane, although in some instances a combination of the two products is targeted. This gas stream is then optionally processed in a CHP engine to generate electricity and heat. Emissions from this process are largely water, with CO2 only being a major component when burning methane. Carbon in this stream is optionally routed to an adjoining synthetic bioreactor to reduce the overall greenhouse gas emissions from the process as a whole. Alternatively, product gas can be flared if no immediate use is available.

The invention may use various aspects of the inventions disclosed and claimed in allowed U.S. patent application Ser. No. 17/863,868 and as shown in FIGS. 1-11 herein and sold under the trademark MADPOWR® (hereafter “MADPOWR Application”), which application has been incorporated in its entirety herein by reference.

These primary and other objects of the invention will be apparent from the following description of the preferred embodiments of the invention and from the accompanying drawings.

### BRIEF DESCRIPTION OF THE DRAWINGS

The following detailed description of the specific non-limiting embodiments of the present invention can be best understood when read in conjunction with the following drawings, where like structures are indicated by like reference numbers.

Referring to the drawings:

FIG. 1A is a plan view of the modular anaerobic digestion system of the MADPOWR Application.

FIG. 1B is another plan view of the System of FIG. 1A.

FIG. 2 is a flow diagram of the invention shown in FIG. 1.

FIG. 3A is a perspective view of the leaching bed of FIG. 1.

FIG. 3B is a cross-section of the leaching bed of FIG. 3A.

FIG. 4A is a perspective view of the liquids tank of FIG. 1.

FIG. 4B is a cross-section of the liquids tank of FIG. 4A.

FIG. 5A is a perspective view of the mixing tank of FIG. 1.

FIG. 5B is a cross-section of the mixing tank of FIG. 5A.

FIG. 6A is a perspective view of the anaerobic digester reactor of FIG. 1.

FIG. 6B is a cross-section of the anaerobic digester reactor of FIG. 6A.

FIG. 7A is a perspective view of the precipitation tank of FIG. 1.

FIG. 7B is a cross-section of the precipitation tank of FIG. 7A.

FIG. 8A is a perspective view of the stripping tank of FIG. 1.

FIG. 8B is a cross-section of the stripping tank of FIG. 8A.

FIG. 8C is a perspective view of the ammonia stripping dropoff of the stripping tank of FIG. 1.

FIG. 8D is a cross-section of the ammonia stripping dropoff of the stripping tank of FIG. 8C.

FIG. 9A is a perspective view of one of the H2S scrubbers of FIG. 1.

FIG. 9B is a cross-section of the H2S scrubber of FIG. 9A.

FIG. 10A is a perspective view of the water remover of FIG. 1.

FIG. 10B is a cross-section of the water remover of FIG. 10A.

FIG. 11 is a plan view of a larger scale modular anaerobic digester system of the MADPOWR Application.

FIG. 12 is a schematic of a waste homogenization system and the components thereof of the invention.

FIG. 13 is a schematic of a feed stock preparation tank and the components thereof of the invention.

FIG. 14 is a schematic of a pretreatment tank and the components thereof of the invention.

FIG. 15 is a schematic of a hydrolysis tank and the components thereof of the invention.

FIG. 16 is a schematic of a photosynthetic bioreactor and the components thereof of the invention.

### DETAILED DESCRIPTION OF THE PREFERRED EMBODIMENTS

The invention is directed to a system for generating renewable energy from common waste streams in a low-carbon manner. This system is modular and applicable to operations of a range of sizes with the goal of enabling its adoption across a wide market. Through use of the system, organic waste is converted into biohydrogen (H2) and/or biomethane (CH4).

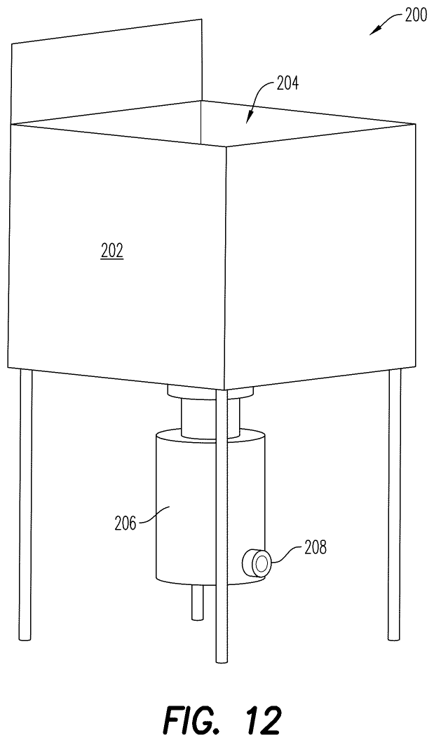

The system will now be discussed in detail and the system's primary components are shown in FIGS. 12-16. Further, the invention may use aspects of the MADPOWR

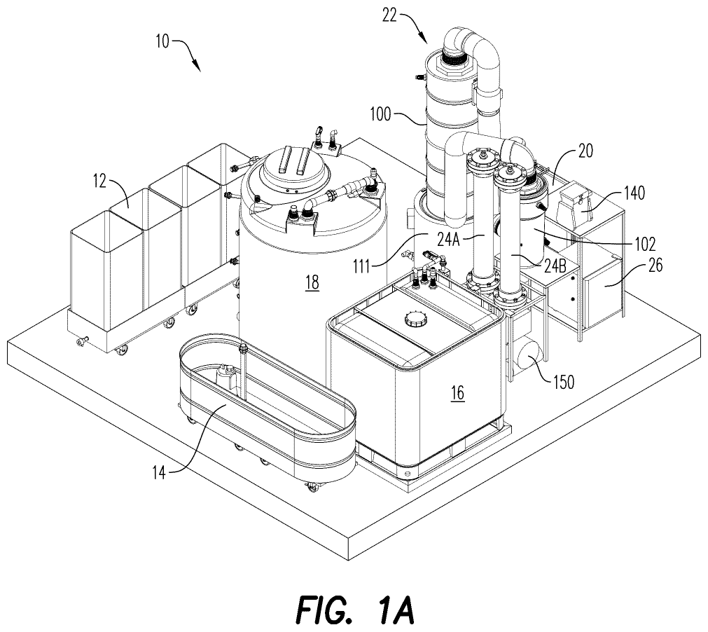

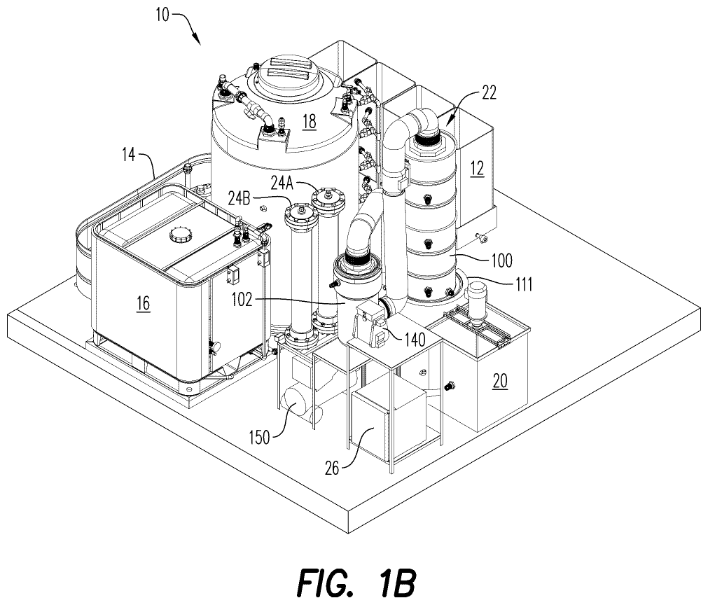

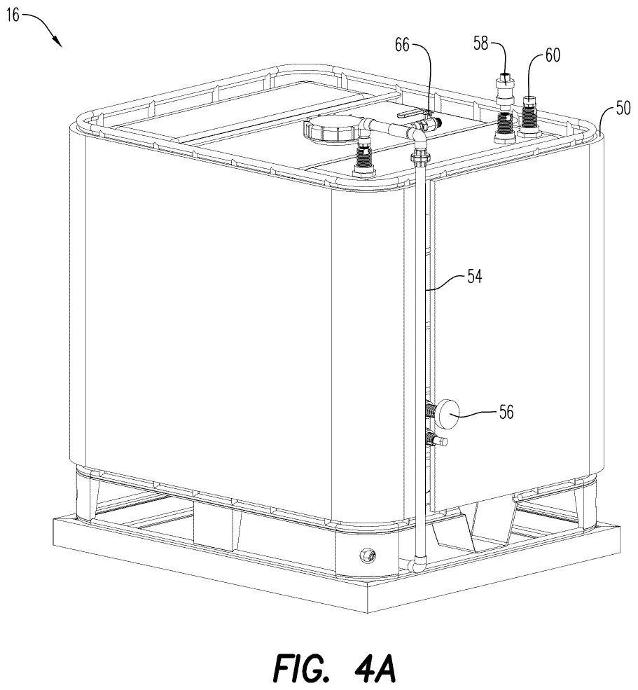

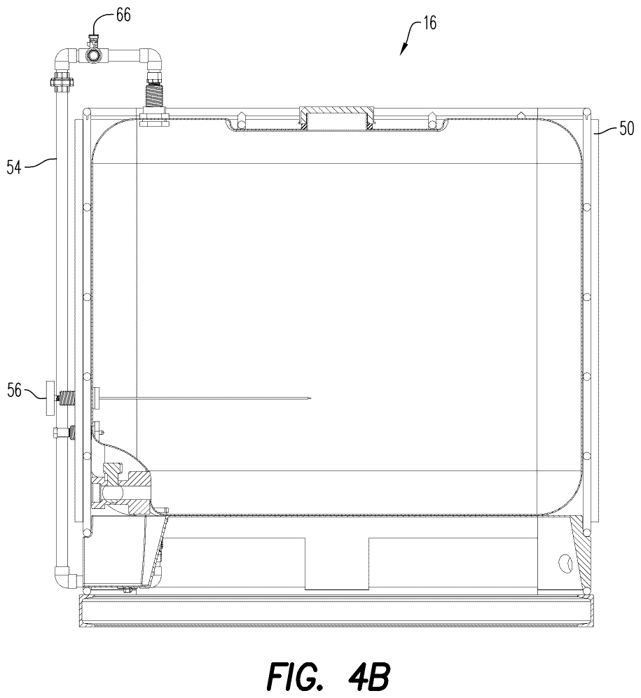

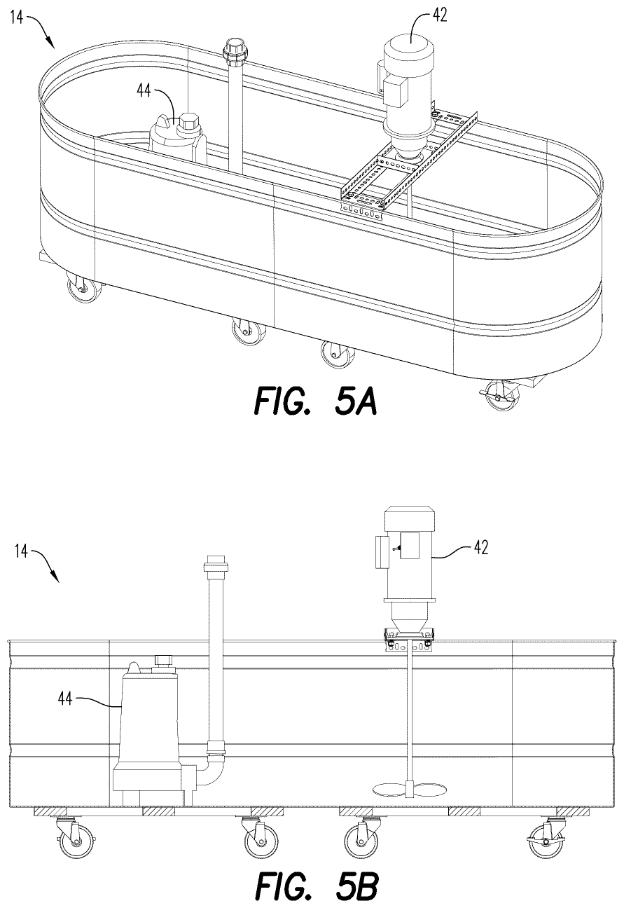

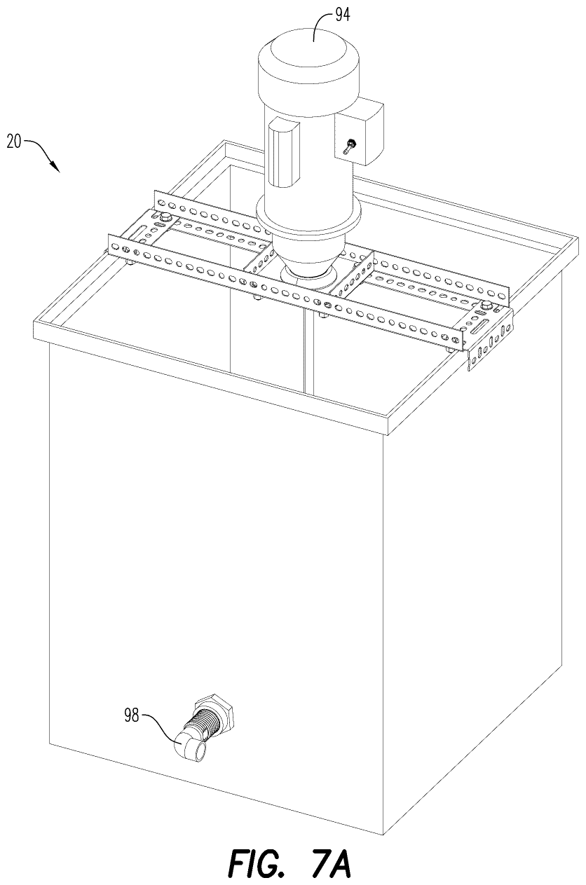

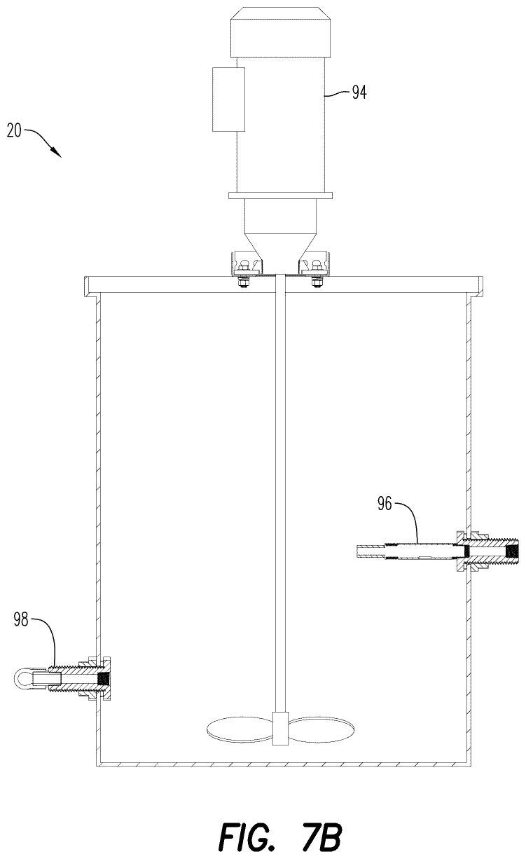

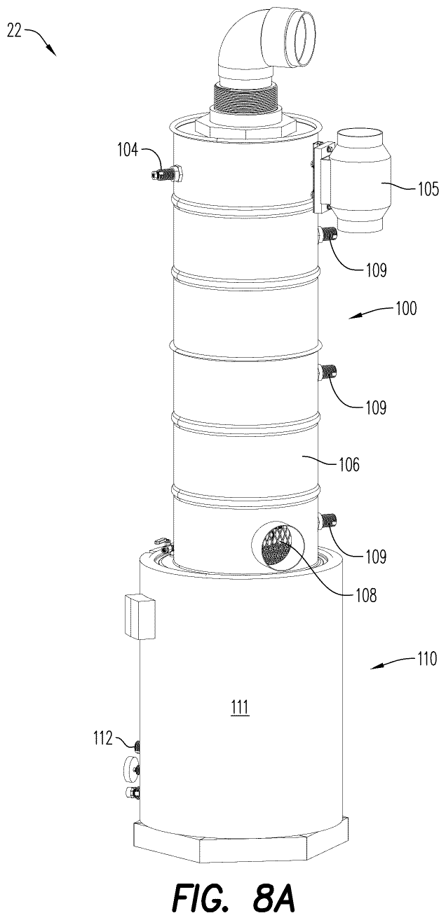

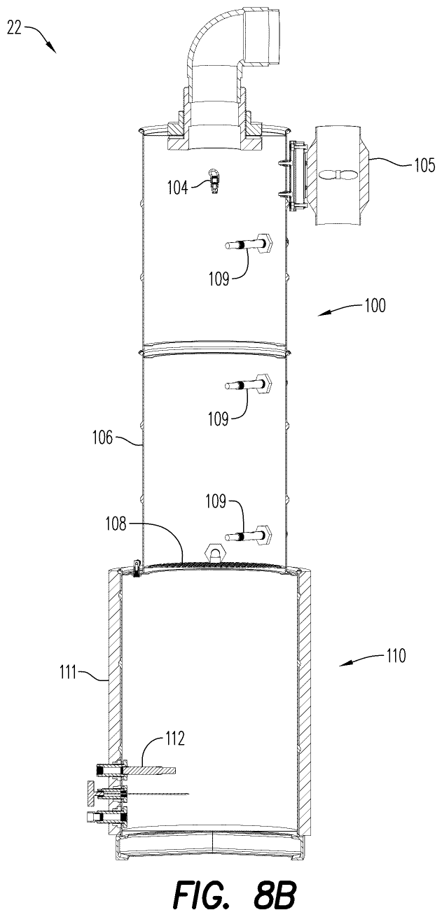

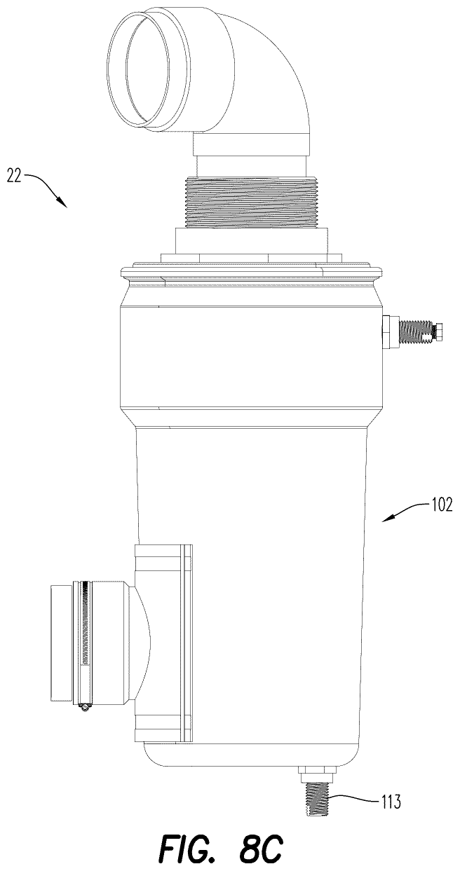

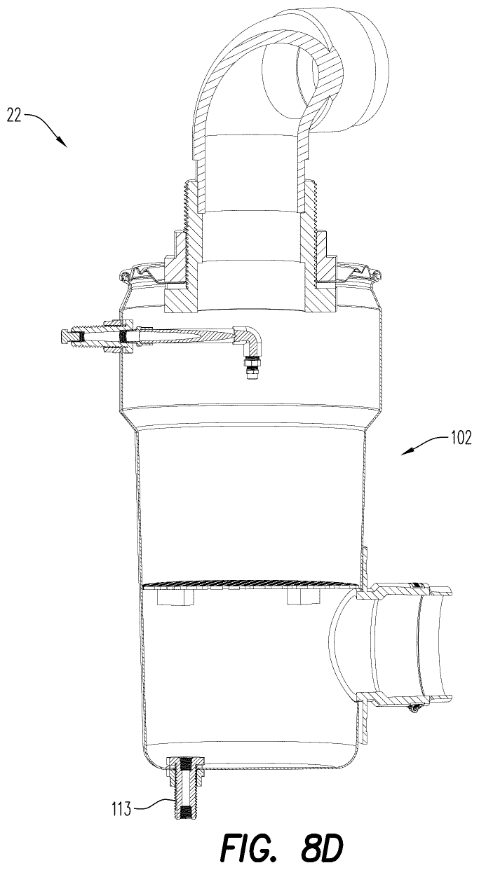

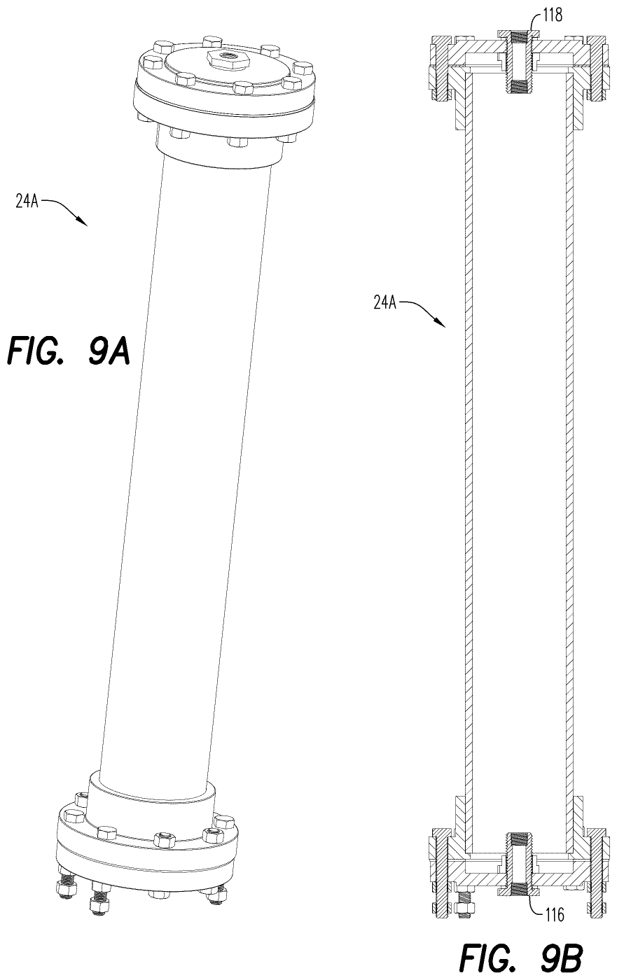

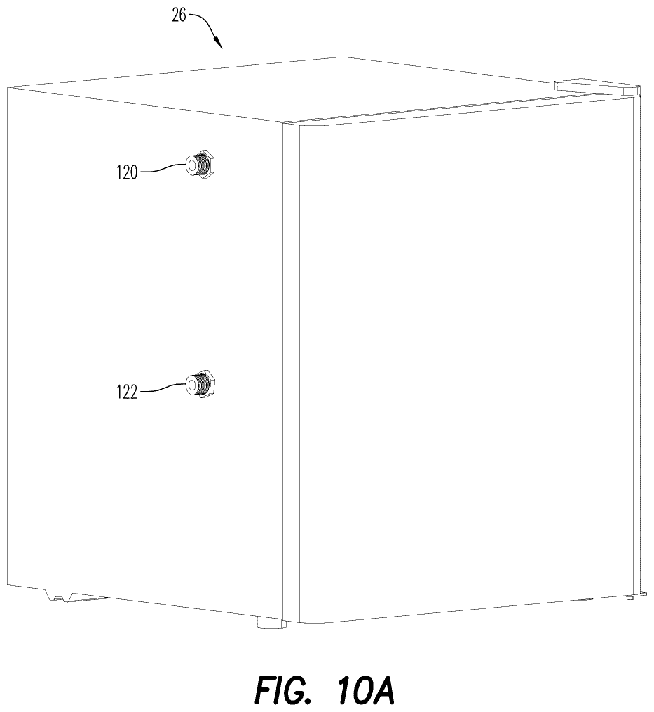

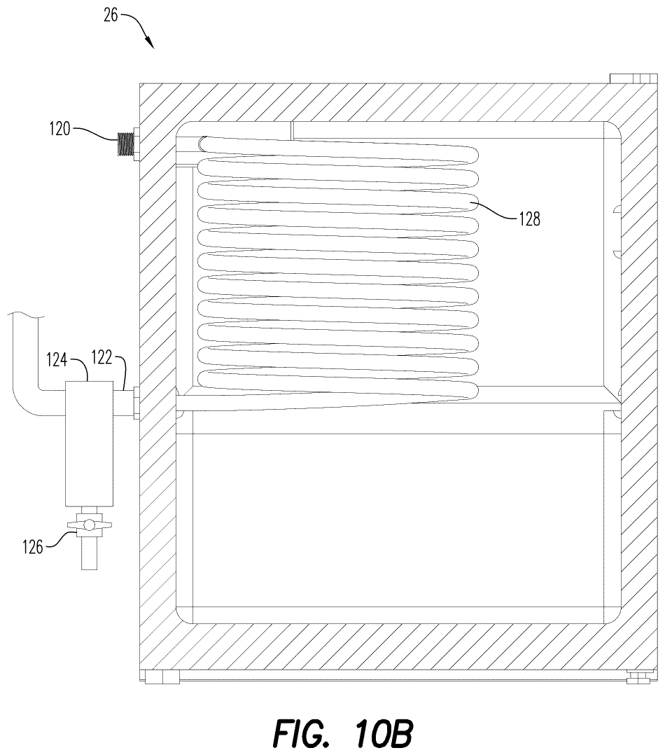

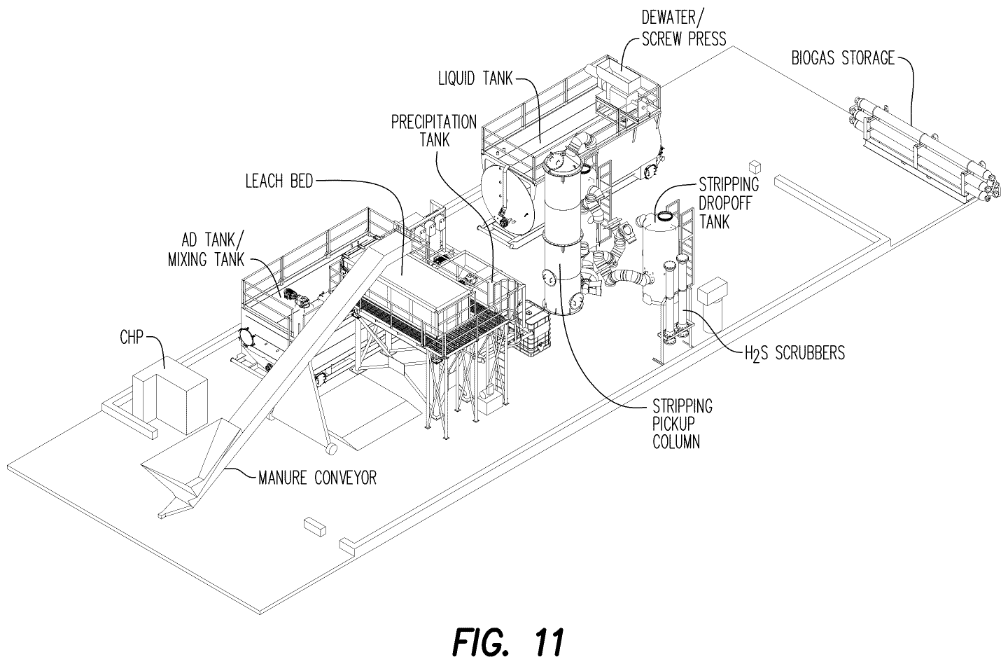

Application shown in FIGS. 1-11. The MADPOWR Application has been incorporated herein by reference and is explained in detail therein, including by the reference numbers in FIGS. 1-11.

Referring to FIG. 12, there is shown the waste homogenization system 200. This component serves to reduce the incoming waste to an approximately consistent size to allow for the subsequent preparation of a relatively homogenous feedstock in a feedstock preparation tank. This is important as a reduced and homogenous particle size is necessary to achieve the most efficient microbial degradation of organic waste. Based on the amount of organic material demanded by the bioreactor component of the system (calculated prior to loading), a proportional amount of waste is fed into the homogenization tank 202 at top 204 which includes one or a combination of macerators, grinders, blenders, mills, etc., e.g. macerator 206, serving to generate a consistent byproduct for transfer to a feedstock preparation tank 220 at outlet 208. Waste is fed in either manually or via a conveyor system from a central waste repository on-site to the homogenization tank.

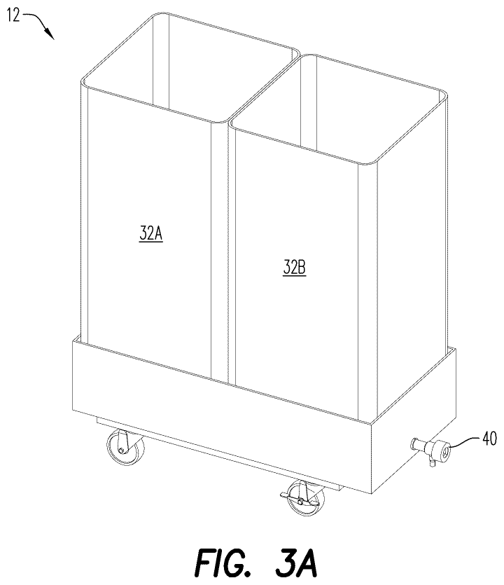

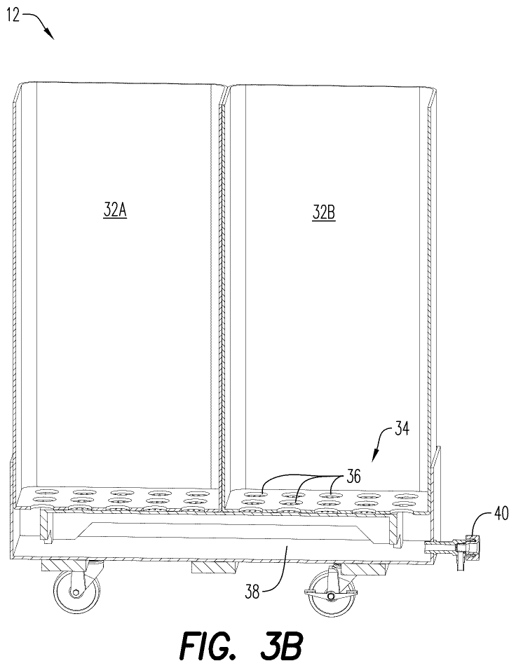

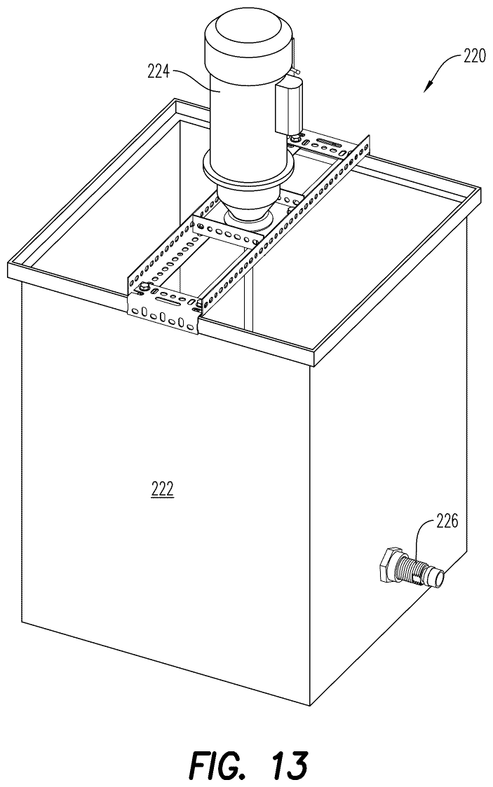

Referring to FIG. 13, there is shown the feedstock preparation tank 220 or the mixing tank. This tank 220 is similar to the mixing tank 14 in the MADPOWR Application and includes a tank 222, rotary mixer 224 and outlet 226. The tank may also include a maceration pump (not shown). This mixing tank receives organic waste that has been previously treated in the homogenization system 200. If, based on laboratory testing, the waste is determined to be nutrient (i.e., NPK content) rich enough to suppress microbial growth, it can be pretreated prior to insertion into the homogenization tank in a leaching bed 12, a precipitation tank 20 and stripping tank 22 as described in the MADPOWR Application and shown, for example, in FIGS. 3A, 7A and 8A. Similarly, if the waste is too low in nutrients, volatile solids, or requires other adjustments to yield a feedstock suitable for microbial decomposition, the addition of supplements or a co-digestible waste stream can be made at this stage. Once the nutrient profile of the waste is properly adjusted, water is added to create a feedstock slurry with a target dilution suitable to allow microbial degradation to occur. After the feedstock is properly diluted, the slurry is mixed to normalize the concentration of dissolved and suspended components across the batch before being pumped into a pretreatment tank 240.

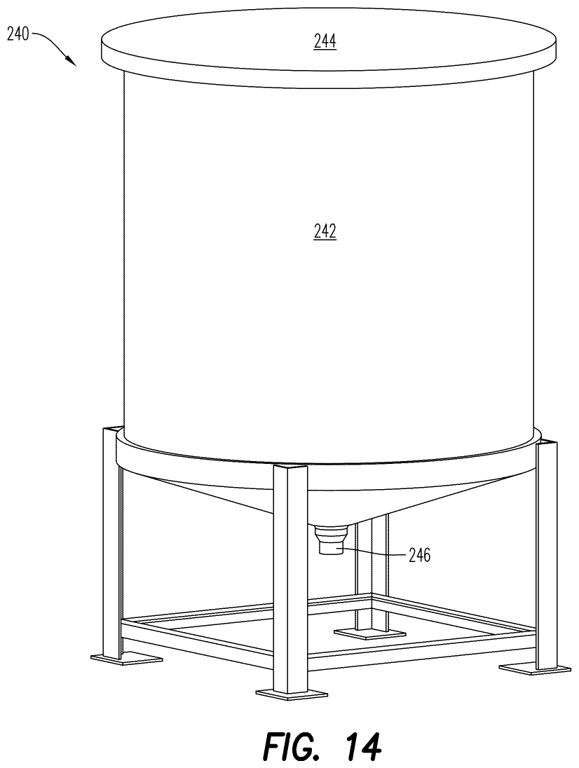

Referring to FIG. 14, there is shown the pretreatment tank 240. The pretreatment tank 240 includes a tank 242, a cover 244, a mixing system (not shown), a heater (not shown), an acid dosing system (not shown) and outlet 246. This tank serves as the primary means of controlling the production of biohydrogen versus biomethane during the process. Here, the slurry is treated with either acid (drop pH to below 6) or heat (temperature above 60° C.) for an extended period of time if biohydrogen is desired. This treatment serves to deactivate methane-producing microorganisms, i.e., methanogens, which are more vulnerable to extreme solution conditions than the sporulating bacteria which can contribute to hydrogen production during the breakdown of waste. If biohydrogen is not desired, this step is skipped altogether, as a mixed culture of microorganisms as found in most waste streams naturally converts organic matter into methane under anaerobic conditions as long as the solution pH and temperature support microbial activity. Once the slurry is properly treated, it is pumped into either an H-tank 260 or the AD 18, respectively.

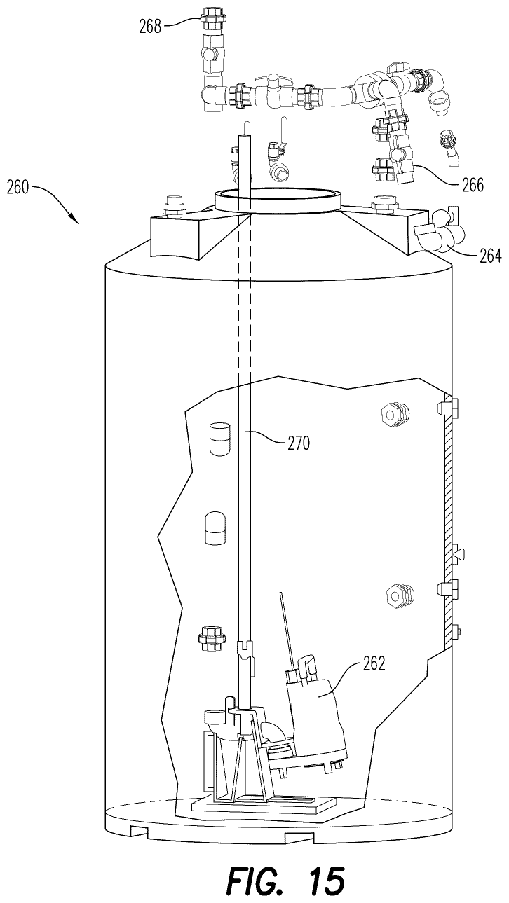

Referring to FIG. 15, there is shown the hydrolysis tank 260. This tank is held at conditions which are suitable to generate biohydrogen from organic waste in the slurry. The most critical requirement to achieve this production is to prevent the growth of methanogens which consume hydrogen while generating methane. To suitably inactivate these microbes, the solution conditions in this tank are maintained at acidic conditions (sub pH 6) which supports microbial activity for many species but precludes methanogens. In addition, the tank is held under anoxic conditions to ensure that waste is degraded following anaerobic rather than aerobic pathways. Once conditions are properly set, the tank is warmed to mesophilic conditions and intermittently mixed to ensure suitable contact occurs between microbes and dissolved organic matter in the slurry. These conditions are held consistent over 3-7 days or continuously if the system is being operated in a continuous mode. Once digestion has been deemed complete, the slurry is either transferred to the anaerobic digestion tank if biomethane is desired in addition to biohydrogen, or directly to the dewatering system if not.

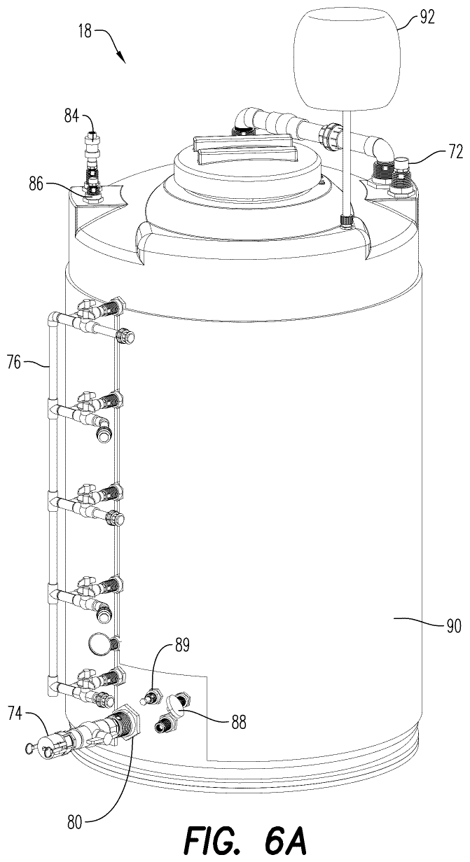

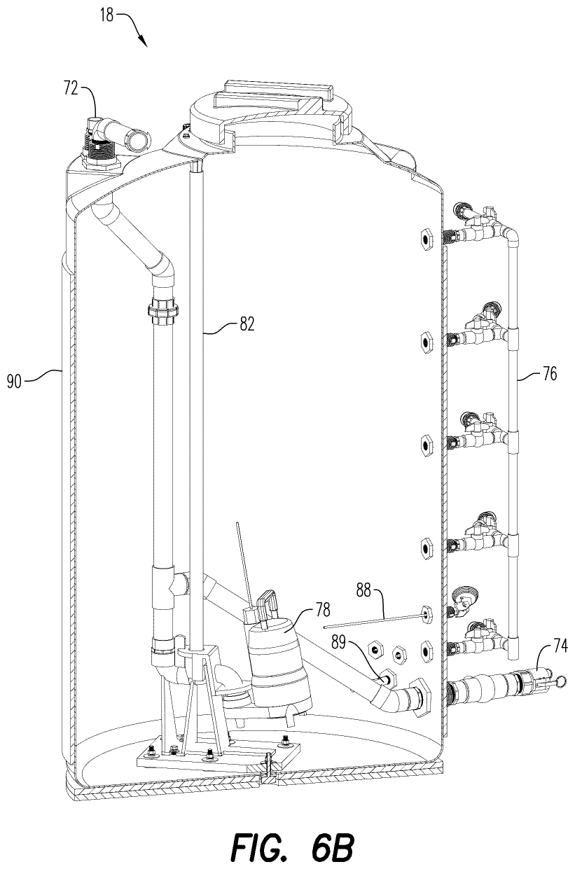

Referring to FIGS. 6A and 6B, there is shown the anaerobic digestion tank 18. If biomethane is desired, feedstock slurry either freshly prepared (without pretreatment) or previously digested in the hydrolysis tank 250 is transferred into the anaerobic digestion tank 18. In contrast to the hydrolysis tank, conditions in this tank are held such that methanogenic growth is supported. This includes mesophilic temperatures, neutral pH, mixing, and anoxic conditions. These conditions are maintained for 21 days, or continuously if fresh feedstock is regularly fed into the tank during continuous operation. If no biomethane is desired, this component can be skipped.

As gas is produced it is transferred through a gas treatment line which consists of a variety of scrubbing systems to condition the product. Such systems may include one or all of a condenser (water removal), iron sponge for hydrogen sulfide (H2S) removal, base (H2S and/or CO2 removal), membranes/water scrubbing/amine scrubbing (CO2 removal), a pressure system (CH4 removal), or other components intended to purify the biohydrogen or biomethane generated in the system. Such systems may be as disclosed in the MADPOWR Application. The exposure of the gas to these various components is selectively chosen based on the target composition of the product fuel. Once gas has been properly treated, it is either used immediately after exiting the line, stored either compressed or at atmosphere, or passed through a CHP or generator to produce electricity and/or heat.

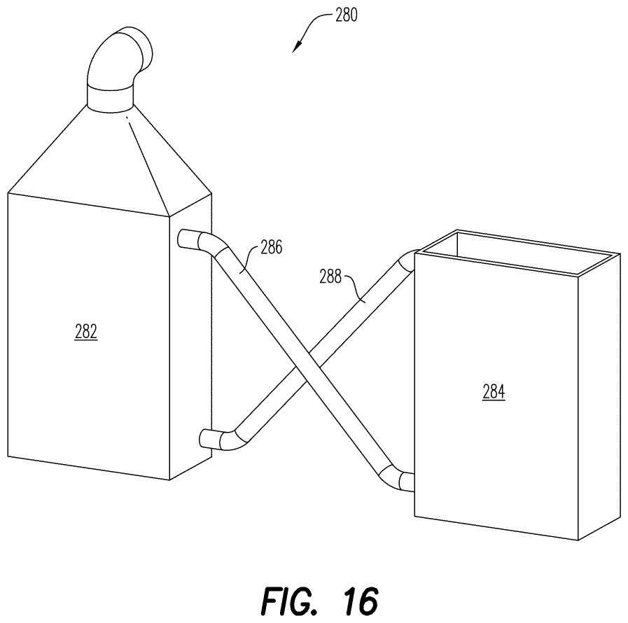

Referring to FIG. 16, there is shown the photosynthetic bioreactor 280 comprising a bubbling tank 282, and algae production tank 284 and piping 286 and 288 which transfers water and CO2 between the two tanks. The photosynthetic bioreactor is a novel means of carbon capture which utilizes photosynthetic algae to naturally sequester CO2 as additional biomass during growth. This component may be considered part of the gas treatment line and is a novel part of the system. Gas from the hydrolysis tank, anaerobic digestor, generator, and/or gas treatment line is bubbled through the photosynthetic bioreactor to remove CO2 from the mixture. Specifically, this carbon is sequestered by the algae growing in the tank which converts it from a gas to a solid. Effectively, this reduces the overall greenhouse gas emissions of the system by cutting the carbon load of emitted gas streams. Furthermore, the produced algal biomass is a nutrient-rich byproduct with uses as fertilizer, animal feed, animal bedding, or subsequent feedstock for digestion in the system.

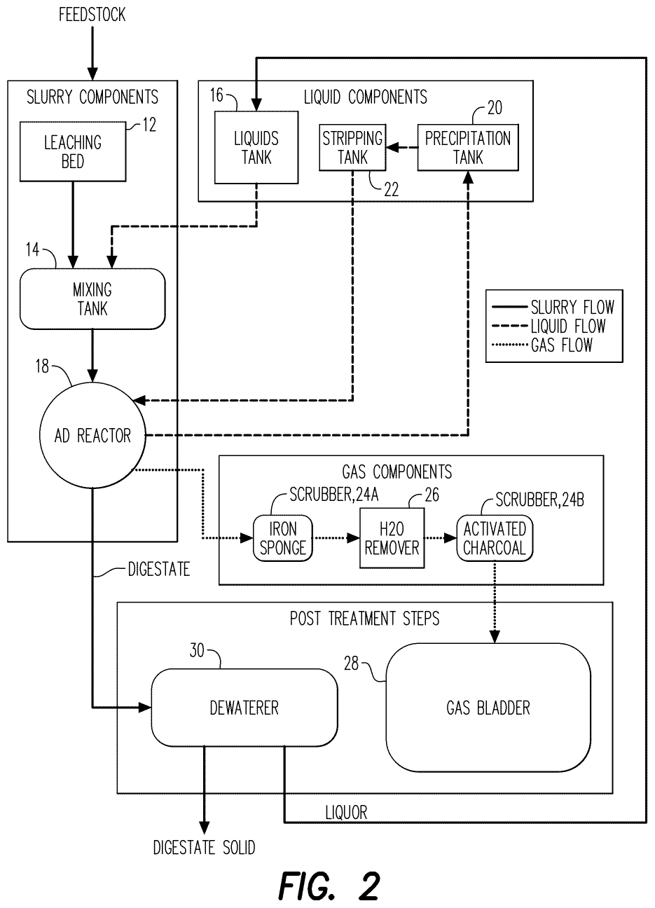

The dewatering system is the terminal treatment for feedstock being processed by the system and serves to separate the solid portion of the slurry from the liquid component. The dewatering system may be as referenced at 30 in FIG. 2 of the MADPOWR Application. Ideally, slurry is only pumped to the dewatering system when its dissolved biodegradable matter has been greatly or entirely diminished via conversion into biohydrogen and/or biomethane. The exact means of dewatering is nonspecific, and can include passive drying, extruding, pressing, squeezing, compressing, heating, pressurizing, and other methods. Separation of the residual solid feedstock of the slurry results in the generation of a nutrient-rich byproduct known as digestate. This digestate is useful as a fertilizer, animal bedding, or marketable byproduct for the user.

Operation of the system begins at the point of waste collection. The organic waste is collected and transported to the system by the user, where it can either be stored, treated, or immediately made into a feedstock slurry. If the chemical composition of the waste is not already known, it should be analyzed by a laboratory to characterize the matter and inform the subsequent treatments needed to yield a digestible feedstock. If the waste is particularly high in potassium, nitrogen, or phosphorus, treatment via leaching, ammonia stripping, and/or precipitation processes may be undertaken. These procedures and components are detailed in the MADPOWR Application. The feedstock is passed through the homogenizing system 200 which can include any one or combination of a macerator, blender, grinder, extruder, crusher, mixer, or other means of particle size reduction as shown in FIG. 12. The homogenized mixture is then pumped into a feedstock preparation tank 220 as shown in FIG. 13 where it is treated to achieve the requisite nutrient content, water content, or digestible matter to support microbial growth. Parameters to consider include % total solids (% TS), % volatile solids (% VS), Carbon: Nitrogen ratio (C/N), total volume, chemical oxygen demand (COD), biological oxygen demand (BOD), volatile fatty acid (VFA) content, or nutrient content. This tank is able to pump prepared feedstock slurry either to (i) the pretreatment tank 240, (ii) the H-tank 260, or (iii) the AD 18.

When targeting the production of biohydrogen, prepared slurry is passed to the pretreatment tank 240 shown in FIG. 14 which is equipped with a mixing system as well as a heater and acid dosing system to drop the pH into the 3-6 range. In some manifestations of the system, this tank may operate concurrently as the H-tank 260 or feedstock preparation tank 220. This tank serves to eliminate methanogens in the waste to ensure that such microorganisms are not able to consume the H2 gas produced during anaerobic fermentation. Elimination of methanogens is achieved through either sustained heating above 60° C. or by dropping the pH of the feedstock via an acid treatment. Conversely, if CH4 is the only desired product and carbon emissions will be captured by the photosynthetic bioreactor, pretreatment of the feedstock is unnecessary.

After pretreatment, feedstock slurry is pumped into the H-tank 260 as shown in FIG. 15 which is a hermetically sealable vessel equipped with various components that may include, but are not limited to, a pumping mechanism 262, a pH control system 264, a gas line 270, gas recirculation 266, and a means of CO2 capture 268. The H-tank 260 contains requisite sampling and monitoring equipment to track and respond to changes in pH, temperature, COD, and gas composition to ensure optimal gas production. Based on these monitors, thermal/pH treatment will be reinitiated when methane levels increase or the H2 partial pressure decreases. After the H-tank is filled, the tank is sealed to foster an oxygen-depleted environment and the pH adjusted to between 3-6 to promote only the growth of non-methanogenic microorganisms. The slurry is then mixed intermittently to ensure thorough contact between the solid and liquid components of the slurry which facilitates fermentation at temperatures between 25-40° C., ideally near 37° C. Throughout the fermentation process, gas primarily composed of H2 is evolved with smaller concentrations of nitrogen (N2), CO2, CH4, carbon monoxide (CO), H2O, and oxygen (O2) potentially present. In normal operation, digestion in the H-tank continues until the H2 production noticeably diminishes as evidenced by evolved volume, gas composition, % VS, COD, or BOD. When such decline is observed, the liquid slurry is extracted from the tank. The volume removed can either be the entirety of the slurry in the H-tank if operating in batch mode, or only a fraction of the volume if operating in continuous mode. In continuous operation, the volume removed is done to balance the hydraulic retention time (HRT), solid retention time (SRT), and organic loading rate (OLR) with respect to the incoming volume of feedstock such that the total volume and digestible matter (i.e., VFA) in the tank remains approximately constant. After the slurry is extracted from the H-tank 260, it is either (i) reintroduced to the H-tank 240 to produce additional H2 if VFAs remain in solution, (ii) pumped to an affixed AD 18 to generate CH4 if VFAs remain in solution, (iii) used to mix in both tanks, (iv) transferred to liquid storage such as a lagoon, (v) directly applied as a fertilizer, or (vi) subjected to a dewatering process.

The anaerobic digestor component is only utilized when biomethane is desired. The system described here is designed to incorporate the MADPOWR AD described in the MADPOWR Application and shown, for example, in FIGS. 6A and 6B. While the AD is not a core piece of the system, a brief description of its incorporation into the process is provided here and in more detail in the MADPOWR Application. Feedstock slurry can be pumped into an adjoining AD tank either from the H-tank 260 after biohydrogen production, or directly from the mixing tank if only a single gas is desired. In contrast to the H-tank 260, the AD 18 aims to promote the growth of methanogens in order to support the production of methane from the feedstock. To achieve this end, the AD is kept at a pH of 7, a temperature of 37° C., and anoxic conditions at all times, as these conditions are ideal for methanogenic growth. A typical hydraulic retention time for the organic matter in the slurry to digest under these conditions is 20-25 days. Gas produced during this time is either recirculated through the slurry to provide mixing or routed to the gas treatment line. Once material is suitably digested, the residual slurry is pumped to the dewatering system.

The gas treatment line is designed to selectively remove gaseous impurities such as hydrogen sulfide, water, siloxane, methane, and carbon dioxide. The final composition of the gas product can be tailored to suit the desired application. In applications which require pure H2, all other gaseous constituents are scrubbed from the product in the treatment line. Similarly, when renewable natural gas (“RNG”) is desired, all components except for CH4 are removed. To support the different treatment regimens required to yield product gases of different profiles, the gas line is equipped with volume meters, pressure gauges, valving, gas composition analyzers, and sampling ports. Such monitoring equipment is installed throughout the line to effectively divide it into sections, enabling the identification of regions which are not operating as intended. The treated gas is then (i) stored either under atmospheric pressure or compressed into a storage tank for subsequent use, (ii) processed in generators or CHP engines to generate heat and/or electricity, (iii) compressed for injection into an RNG or H2 pipeline, (iv) recirculated back to the H-tank to facilitate mixing, or (v) flared or vented should no alternative be viable.

The system utilizes a novel means of CO2 removal by incorporating a photosynthetic bioreactor as shown in FIG. 16 which can be affixed to the gas treatment line after other adulterant species have been removed from the stream. This innovative approach to carbon capture contributes to the reduction of the systems operational carbon footprint. The bioreactor comprises an algae culture, a substrate for algal proliferation, water with controlled pH and nutrient content, managed dissolved oxygen levels, a sunlight-permeable barrier for photosynthesis, and a system for gas sparging and recovery. Gas from the treatment line is sparged into the bioreactor to increase the availability of CO2 to the algae, thus enhancing its capture as additional biomass via photosynthesis. Algal biomass serves a secondary purpose as animal feed or fertilizer due to its rich nutrient profile. Emissions from generators, CHPs, heaters, or other components of the system or installation site can also be diverted to the bioreactor for further mitigation of carbon footprint.

If no further digestion of the feedstock is feasible or desired, it may be applied as a fertilizer either as a slurry or following a drying process to produce a product known as digestate. This drying is achieved by pumping the spent slurry to a dewatering system which can consist of a variety of methods including mechanical systems (e.g., screw press), passive drying, windrowing (i.e., air drying), solid-liquid separation (e.g., flocculating), screening, or centrifugation. Once dried, the digestate can be stored, transported off site, or applied to fields as a fertilizer.

The system with components interconnected as described represents a means to convert organic waste into renewable energy via the generation of biohydrogen and/or biomethane in a low-carbon manner. This is the only known system providing a combination of the apparatus, scalability, waste handling, interchangeable biohydrogen and biomethane production, renewable energy, and a means of carbon reduction via the capturing of emissions via the use of a photosynthetic bioreactor.

In contrast to the prior art, there is shown a system which is simple to use and accessible to users regardless of company size, training, or resource availability. To achieve this, the system uses naturally occurring, non-modified, mixed anaerobic cultures found in typical AD feedstocks (e.g., organic waste such as manure, crops, food, wastewater, textiles, plant matter, etc.), thus allowing its operation even by users without microbiological expertise. In addition, the system is easily constructed as shown in the Figures herein and operation is possible without expertise, including the ability to control the relative degrees of biohydrogen and biomethane produced.

The invention is a multifunctional system which transforms organic waste into carbon-free renewable energy, making it uniquely applicable to the domains of sustainability, waste management, and energy generation. By facilitating the conversion of a wide array of organic waste materials into biohydrogen and biomethane, the invention addresses the pressing need for effective waste reduction strategies while simultaneously advancing the accessibility of clean renewable energy. Its versatile sizing and ability to handle a variety of waste streams promotes its use by businesses who need a solution for disposing of their organic waste in a low carbon manner. In addition to providing a solution for handling low-value waste streams, the system also grants the user a source of renewable energy including energy, heat, H2, and CH4. The biohydrogen generated through this process is a carbon-neutral fuel, emitting only water upon combustion, thereby contributing significantly to the reduction of greenhouse gas emissions and the mitigation of climate change. Additionally, the production of biomethane offers a sustainable alternative to natural gas, further diversifying energy resources and enhancing energy security. Incorporating a photosynthetic bioreactor for carbon dioxide sequestration extends the environmental benefits of the system by actively reducing the carbon footprint associated with energy generation from organic waste. This aspect of the invention not only exemplifies its alignment with global sustainability objectives but also enhances its commercial viability by offering a method to achieve carbon neutrality or even negativity in energy production processes. The generated renewable energy and carbon reductions may also enable users to qualify for renewable energy incentives, thus adding additional financial benefit to its operation as a whole. Moreover, the invention promotes resource efficiency by converting waste materials into valuable sources of energy and other byproducts such as digestate which can be utilized as a natural fertilizer, thereby closing the loop in organic waste management. Fertilizer streams produced from system operation (digestate, potassium from leaching, nitrogen from stripping, phosphorus from precipitation, algal biomass) can be used independently or recombined in specific proportions to make a single fertilizer product with a customizable nutrient profile. This practice is important for organizations and businesses such as restaurants and farms which may benefit from their environmental stewardship and awareness and utilize the fertilizer streams to grow their own crops. This approach not only minimizes the environmental impact associated with waste disposal but also aligns with the principles of the circular economy.

In summary, the invention provides a comprehensive solution that significantly contributes to the reduction of environmental pollution, fosters the generation of renewable energy, and offers economic benefits through cost savings and potential revenue generation from byproducts. Its implementation will contribute to the advancement of sustainable development goals, marking a significant leap forward in the fields of waste management and renewable energy production.

The system exhibits remarkable adaptability, a core attribute that enhances its applicability across a range of different waste-to-energy projects. Its base configuration is suitable for processing conventional low-nutrient feedstocks such as manure or food waste to generate biohydrogen. The modularity of the system makes the addition and removal of components along both the liquid and gas lines simple, including facile rerouting of process streams through the actively used/installed system components without reliance on the unneeded portions. This also allows for a pared down system to be constructed at installation as components that are not immediately required can be added at a later date. The system can be equipped with leaching, stripping, and precipitation components to treat feedstocks which are rich in nutrients (e.g., poultry manure) to reduce their nutrient levels to better suit microbial growth and metabolism, but these components can be avoided with feedstocks which do not require adjustment. The system also hosts the capability to process organic waste streams of disparate profiles via maceration, dilution, and other adjustments to yield a viable feedstock as. To further reduce the carbon footprint of the system, an optional photosynthetic bioreactor can be incorporated to enable carbon sequestration. Moreover, for operations which desire biomethane in addition to biohydrogen, an AD module can be readily incorporated into the system. Depending on the particular composition of product gas desired, the gas treatment line can consist of various scrubbing components to remove any or all of contaminant gases including water, H2S, CO2, siloxanes, CH4, etc. All discussed components are scalable to manage both small- and large-scale operations. The versatility of the system enables it to utilize different types of organic waste, microbes, uses for generated gases, uses for energy produced, uses for heat produced, other types of mixing, pumping, drying, changes in material, sizing, volumes, amounts, pressure, time, and other critical process parameters. This comprehensive adaptability positions the system as a versatile solution in the realm of waste-to-energy conversion, capable of meeting a broad spectrum of operational needs and environmental goals.

The system operates on well-established scientific principles and technical methodologies. Under anoxic conditions, consortia of anaerobic microorganisms are able to convert organic waste into methane in a process known as anaerobic digestion. This four-step biochemical process entails the degradation of complex molecules such as polysaccharides, proteins, and biopolymers into their fundamental building blocks such as sugars, amino acids, and other small molecules. The first three steps of the process are the hydrolysis, acetogenesis, and acidogenesis steps which result in the production of VFAs, acetate, and H2 and CO2 gas as byproducts. The final step of the process is known as methanogenesis and involves the production of CH4 from the byproducts of the initial three steps. This CH4 production proceeds via either mineralization of VFAs/acetate or via the biochemical combination of H2 and CO2. Both of these pathways rely upon the natural metabolic activity of a class of archaea known as methanogens to proceed. By inhibiting the activity of methanogens, byproducts of the first three stages of anaerobic digestion, including H2, accumulate which leads to H2 and CO2 becoming the preeminent gas phase product species of the process.

To achieve this inhibition, the system takes advantage of the thermal and acidic sensitivity of methanogen. Conversely, certain sporulating microorganisms which are capable of initiating the fermentation of organic waste remain viable under these conditions, thus allowing the early stages of anaerobic digestion and H2 production to proceed without completing conversion to methane. To ensure the maximal generation of H2, incoming feedstock and the environment within the H-tank are maintained at a low pH or subjected to periodic temperature elevations, thereby preventing methanogen resurgence from insufficiently treated feedstock. Any recurrence of methanogen growth is signified by a related increase in CH4 and decrease in H2 in the product gas. When such a trend is observed, a renewed treatment of the feedstock slurry is performed. Such tending to the process allows for a gas product primarily consisting of H2 and CO2 to be output from the H-tank. This gas stream is purified to biohydrogen by removing CO2 and other trace contaminants, a treatment for which there are a number of options available. The preferred means of CO2 removal in the system is via the utilization of a photosynthetic bioreactor in order to maximize the sustainability of the system. The bioreactor operates using photosynthetic algae which can utilize CO2, sunlight, water, and nutrients to generate additional biomass, thus removing carbon from the gas phase in a carbon capture process (i.e., gas->solid conversion). This effectively removes carbon emissions from the process and simultaneously generates useful byproducts in the form of biohydrogen and nutrient-rich algae. Thus, this component not only affirms the system's efficacy in carbon sequestration but also contributes to a circular approach to waste and emission management.

After H2 production, it is possible that some VFAs— the immediate precursors to acetate in the anaerobic digestion process-remain in the slurry after H2 production slows due to an imperfect digestion process. Slurry still containing VFAs can either be recirculated for additional H2 generation or transferred to an AD containing active methanogens to support CH4 production. The use of an AD permits the generation of CH4 in addition to the H2 that was already evolved in the H-tank, thus achieving the dual-fuel capability of the system. This also permits the controlled loading of VFAs into the AD which greatly facilitates the maintaining of the neutral pH required to support methanogen activity. In addition, given the prior degradation of the organic material, CH4 production is expedited in the AD compared to a full fermentation cycle. When only CH4 is desired, no acid or heat pretreatment is necessary, and the system can operate as a traditional AD.

This flexibility underscores the system's capacity to produce either H2, CH4, or both gases sequentially. While both product gases are potential sources of renewable energy, the environmental virtue of H2 as a fuel lies in its combustion product-water-being carbon-free. When the majority of dissolved VFAs in the slurry have been exhausted, other nutrients initially found in the feedstock still remain in solution. These can be harnessed as a fertilizer by applying the digested slurry to fields as a liquid, or dewatering into a more concentrated solid nutrient source in the dewatering system.

Upon receipt of organic waste, analytical determinations of nutrient content such as nitrogen, phosphorus, potassium, metals, etc. is performed. In feedstocks with high potassium and/or phosphorus, leaching is recommended, and in feedstocks with high nitrogen content, stripping is recommended following the method outlined in the MADPOWR Application. Once the waste has been adjusted to the appropriate nutrient profile, analytical determination of the % TS, % VS, COD, and BOD is performed using standard protocols such as ASTM E1756-08, D1252-06, and WK23808. Based on these analyses, the feedstock is diluted to optimize parameters such as % TS, % VS, and C/N ratio which vary depending on the specific feedstock characteristics. Here, food waste is being digested which has an initial % TS of 22%, % VS of 90%, adequate nutrients to support microbial activity, and an ideal C/N ratio of 25:1. This waste is ideally suited for biohydrogen production, and thus no co-digestion or pre-conditioning is required before introduction to the homogenization system. This homogenized waste is then transferred to the mixing tank and diluted to a &TS of 5-10%. For a system iteration equipped with a 500-gallon tank, the feedstock is diluted to a slurry of roughly 8% TS by adding approximately 360 gallons of water to 336 lbs. of food waste and mixing thoroughly. The slurry is then pumped into the pretreatment tank where it is treated by the heating system at 80° C. for 1.5 hours before cooling back to 25-40° C. Once cool, the slurry is transferred into the H-tank which is then sealed, heated to ˜37° C., and mixed intermittently (every 4 hours). Over the first few days of this process, gas is evolved and the pH of the slurry decreases as the acidogenesis phase of anaerobic digestion proceeds. The composition of this gas is tracked using the sampling ports in the tank. Before the pH drops into the 3-6 range, this gas will be a mix of H2, CO2, and CH4 as is expected prior to deactivation of methanogens in the waste. However, once acidic conditions are reached and maintained, the methanogens in the slurry naturally begin to fade, causing the gas to enrich in H2 and CO2 rather than CH4. Any recurrence of methanogen growth is indicated by an enrichment of the gas with CH4 which prompts subsequent acid or heat treatment. Once the gas production stabilizes, operation of the system in continuous mode begins involving the daily replacement of 20 gallons of slurry with fresh feedstock prepared in the same way as the initial loading to provide an HRT of 25 days. The extracted slurry is either passed to an AD system or to a dewatering system. After dewatering, the digestate is applied to land as a fertilizer or stored for later use.

Gas purification passively occurs along the gas treatment line and is scrubbed of contaminant gases. The operator monitors the media of each unit of the treatment system (e.g., iron scrubber media, water removal system, algae health and nutrients/pH of media) daily to verify that the gas is still being treated properly. This is also apparent when sampling the evolved gas throughout the gas treatment line to ensure that each contaminant (e.g., H2S, siloxanes, CO2, etc.) is being suitably removed. Treated gas is then either compressed for storage, burned in a generator, or flared to generate a low-carbon emittant if no present use exists. With these described practices, the system can continuously operate, digest food waste, and generate biohydrogen as long as waste continues to be delivered to the site and resources such as water and electricity are available for use.

The exemplary embodiments herein disclosed are not intended to be exhaustive or to unnecessarily limit the scope of the invention. The exemplary embodiments were chosen and described in order to explain the principles of the present invention so that others skilled in the art may practice the invention. As will be apparent to one skilled in the art, various modifications can be made within the scope of the aforesaid description. Such modifications being within the ability of one skilled in the art form a part of the present invention and are embraced by the appended claims.

1. A modular point-of-waste to renewable energy system for converting organic feedstock material to biohydrogen and optionally biomethane (“System”) comprising
a waste homogenization tank adapted to receive the organic feedstock material and adapted to reduce the feedstock material to an approximately consistent size to provide a homogenized feedstock material,
a feedstock preparation tank adapted to receive the homogenized feedstock material from the waste homogenization tank and adapted to mix the homogenized feedstock material with a liquid to provide a feedstock slurry,
a pretreatment tank adapted to receive the feedstock slurry from the feedstock preparation tank and adapted to treat the feedstock slurry with acid and/or heat,
a hydrolysis tank adapted to receive the treated feedstock slurry from the pretreatment tank and adapted to generate biohydrogen from the treated feedstock slurry,
a gas treatment line adapted to receive the biohydrogen from the hydrolysis tank and adapted to treat the biohydrogen to remove other components from the biohydrogen and having a termination to exit the biohydrogen for subsequent use,
optionally an anaerobic digester reactor adapted to receive the treated feedstock slurry and adapted to generate biomethane, and
optionally a photosynthetic reactor including a photosynthetic algae adapted to receive gas from the hydrolysis tank, the anaerobic digester reactor and/or the gas treatment line and adapted to remove CO2.

2. The System according to claim 1 wherein the System includes an anaerobic digester reactor adapted to receive the treated feedstock slurry and adapted to generate biomethane.

3. The System according to claim 1 wherein the System includes a photosynthetic reactor including a photosynthetic algae adapted to receive gas from the hydrolysis tank, the anaerobic digester reactor and/or the gas treatment line and adapted to remove CO2.

4. The System according to claim 2 wherein the System includes a photosynthetic reactor including a photosynthetic algae adapted to receive gas from the hydrolysis tank, the anaerobic digester reactor and/or the gas treatment line and adapted to remove CO2.

5. The System according to claim 1 wherein the pretreatment tank comprises a tank, a cover, a mixer, a heater and an acid dosing system.

6. The System according to claim 1 wherein the photosynthetic reactor comprises a bubbling tank, an algae production tank and first and second piping adapted to transfer water and CO2 between the bubbling tank and the algae production tank.

7. A modular point-of-waste to renewable energy system for converting organic feedstock material to biomethane (“System”) comprising
a waste homogenization tank adapted to receive the organic feedstock material and adapted to reduce the feedstock material to an approximately consistent size to provide a homogenized feedstock material,
a feedstock preparation tank adapted to receive the homogenized feedstock material from the waste homogenization tank and adapted to mix the homogenized feedstock material with a liquid to provide a feedstock slurry,
an anaerobic digester reactor adapted to receive the feedstock slurry from the feedstock preparation tank and adapted to generate biomethane,
a gas treatment line adapted to receive the biomethane from the anaerobic digestor reactor and adapted to treat the biomethane to remove other components from the biomethane and having a termination to exit the biomethane for subsequent use, and
optionally a photosynthetic reactor including a photosynthetic algae adapted to receive gas from the anaerobic digester reactor and/or the gas treatment line and adapted to remove CO2.

8. The System of claim 7 wherein the System includes a photosynthetic reactor including a photosynthetic algae adapted to receive gas from the anaerobic digester reactor and/or the gas treatment line and adapted to remove CO2.

9. The System of claim 8 wherein the photosynthetic reactor comprises a bubbling tank, an algae production tank and first and second piping adapted to transfer water and CO2 between the bubbling tank and the algae production tank.

10. A method of treating an organic feedstock at a point-of-waste location to convert the organic feedstock to biohydrogen and optionally biomethane comprising the steps of
a. providing the organic feedstock to a modular system comprising
a waste homogenization tank adapted to receive the organic feedstock material and adapted to reduce the feedstock material to an approximately consistent size to provide a homogenized feedstock material,
a feedstock preparation tank adapted to receive the homogenized feedstock material from the waste homogenization tank and adapted to mix the homogenized feedstock material with a liquid to provide a feedstock slurry,
a pretreatment tank adapted to receive the feedstock slurry from the feedstock preparation tank and adapted to treat the feedstock slurry with acid and/or heat,
a hydrolysis tank adapted to receive the treated feedstock slurry from the pretreatment tank and adapted to generate biohydrogen from the treated feedstock slurry,
a gas treatment line adapted to receive the biohydrogen from the hydrolysis tank and adapted to treat the biohydrogen to remove other components from the biohydrogen and having a termination to exit the biohydrogen for subsequent use,
optionally an anaerobic digester reactor adapted to receive the treated feedstock slurry and adapted to generate biomethane, and
optionally a photosynthetic reactor including a photosynthetic algae adapted to receive gas from the hydrolysis tank, the anaerobic digester reactor and/or the gas treatment line and adapted to remove CO2,
b. treating the organic feedstock in the modular system, and
c. obtaining biohydrogen and optionally biomethane.

11. The method according to claim 10 wherein the modular system includes an anaerobic digester reactor adapted to receive the treated feedstock slurry and adapted to generate biomethane.

12. The method according to claim 10 wherein the modular system includes a photosynthetic reactor including a photosynthetic algae adapted to receive gas from the hydrolysis tank, the anaerobic digester reactor and/or the gas treatment line and adapted to remove CO2.

13. The method according to claim 11 wherein the modular system includes a photosynthetic reactor including a photosynthetic algae adapted to receive gas from the hydrolysis tank, the anaerobic digester reactor and/or the gas treatment line and adapted to remove CO2.

14. The method according to claim 10 wherein the pretreatment tank comprises a tank, a cover, a mixer, a heater and an acid dosing system.

15. The method according to claim 10 wherein the photosynthetic reactor comprises a bubbling tank, an algae production tank and first and second piping adapted to transfer water and CO2 between the bubbling tank and the algae production tank.
